(19) United States
(12) Patent Application Publication
(10) Pub. No.: US 2007/0167836 A1
(43) Pub. Date: Jul. 19, 2007

(54) Multi modal spectroscopy

(75) Inventors: Obrad Scepanovic, Cambridge, MA (US); Joseph Gardecki, Acton, MA (US); Michael S. Feld, Jamaica Plain, MA (US)

Correspondence Address: WEINGARTEN, SCHURGIN, GAGNEBIN & LEBOVICI LLP, TEN POST OFFICE SQUARE, BOSTON, MA, 02109, US

(73) Assignee: Massachusetts Institute of Technology

(21) Appl. No.: 11/492,301

(22) Filed: Jul. 25, 2006

Related U.S. Application Data
(60) Provisional application No. 60/702,248, filed on Jul. 25, 2005.

(51) Int. Cl. A61B 6/00

(52) U.S. Cl. 600/476

(57) ABSTRACT

The present invention relates to multimodal spectroscopy (MMS) as a clinical tool for the in vivo diagnosis of disease in humans. The MMS technology combines Raman and fluorescence spectroscopy. A preferred embodiment involves diagnosis cancer of the breast and of vulnerable atherosclerotic plaque, esophageal, colon, cervical and bladder cancer. MMS is used to provide a more comprehensive picture of the metabolic, biochemical and morphological state of a tissue than afforded by either Raman or fluorescence and reflectance spectroscopies alone.

### CROSS REFERENCE TO RELATED APPLICATION

This application claims the priority of U.S. Provisional Application No. 60/702,248, filed Jul. 25, 2005 entitled, MULTI MODAL SPECTROSCOPY. The entire content of the above application is being incorporated herein by reference.

### BACKGROUND OF THE INVENTION

Techniques capable of evaluating human disease in a safe, minimally invasive and reproducible way are of importance for clinical disease diagnosis, risk assessment, therapeutic decision-making, and evaluating the effects of therapy, and for investigations of disease pathogenesis and pathophysiology. Among the clinical methods available to diagnose tissue lesions, pathologic examination of cytology preparations, biopsies and surgical specimens is the present day standard.

Pathologists have traditionally based their diagnoses primarily on tissue morphology. However, as the field of diagnostic pathology has evolved, assessment of tissue morphology has become more sophisticated, including such techniques as morphometry (or quantitative image analysis) and ploidy analysis. Pathologic diagnosis has also begun to move from complete dependence on morphology to inclusion of a host of adjunct techniques that provide biochemical and molecular information as well. This is particularly true for the diagnosis of cancer, where routine diagnosis begins with morphology but usually also includes such molecular diagnostic techniques such as immunohistochemistry and in situ hybridization that identify specific molecular signatures.

This molecular information is not only of use for diagnosis, but is also of use for risk assessment and therapeutic decision-making, for example, in qualifying patients for molecular therapies, such as gene therapy or therapy with monoclonal antibodies directed against specific molecular targets. This molecular information has also greatly advanced the understanding of the pathogenesis and pathophysiology of many diseases, particularly cancer. But this evolution toward a focus on molecular events is not unique to the diagnosis of cancer. Recent molecular studies are also beginning to shed light on the pathogenesis and pathophysiology of cardiovascular disease, not only atherosclerosis but other disease (such as the cardiomyopathies) as well.

### SUMMARY OF THE INVENTION

Diseases are more reliably identified by biochemical signatures than purely morphological markers. The present invention relates to the use of Raman spectroscopy in combination with other spectroscopic methods to provide biochemical and morphologic information and to further provide molecular information reflective of the metabolic state of tissue. This combination of biochemical, morphologic and metabolic information is used as the basis of more robust diagnostic methods. These types of molecular signature can be used for disease diagnosis, the disease progression and response to therapy.

Thus, in a preferred embodiment Raman and fluorescence can be used in combination to measure tissue in vivo using a probe or can be used to measure excised tissue samples. In a further embodiment Raman and reflected light can be used in combination for measurements on a human or animal body with a probe or on biological samples. Additionally, Raman, fluorescence and reflectance measurements can be made using a probe for in vivo or ex vivo measurements. A common light delivery and light collection probe can be used in preferred embodiments of the invention.

The combination of modalities in the modal spectroscopy (TMS) has several advantages over the single modalities alone. First, fluorescence spectroscopy provides information about tissue metabolites, such and NADH, that are not provided by Raman spectroscopy. Second, TMS uses diffuse reflectavi spectroscopy (DRS) to overcome distortion of fluorescence signatures by the effects of tissue absorption and scattering, and extract the intrinsic fluorescence signature (IFS). Third, in addition to its value in extracting IFS, DRS provides critical information about the tissue absorbers and scatterers themselves. Finally, while DRS provides information about tissue components responsible for diffusive scattering, light scattering spectroscopy (LSS) provides information about tissue components responsible for single backscattering. The combination of techniques into TMS, therefore, provides a wealth of information about tissue fluorophores, absorbers and scatterers, which creates a much more complete biochemical, morphologic and metabolic tissue profile.

TMS and Raman methods have been applied to specific diseases based on the strengths of each spectroscopic modality for detecting the primary biochemical or morphologic hallmarks of that disease. For example, cancer is a characterized by rapid cellular proliferation that is reflected in increased cellular metabolism. TMS, which provides IFS and DRS information about key cellular metabolites such as NADH and oxy- and deoxy-hemoglobin is, thus, a natural choice of modality for the diagnosis of cancer. TMS also provides information about key morphologic cellular changes, such as the nuclear enlargement and pleomorphism (variation in size and shape), that are characteristic of cancer. On the other hand, vulnerable atherosclerotic plaque is the end result of an inflammatory process that leads to thinning and rupture of the fibrous cap, leading to the release of thrombogenic necrotic lipid core material into the blood stream. Atherosclerotic plaques are also subject to calcific mineralization of the fibrous cap and necrotic core. Most lipids and calcium salts are strong Raman scatterers and, thus, Raman spectroscopy is a natural choice of modality for the diagnosis of vulnerable atherosclerotic plaque.

The combination of spectroscopic modalities in multimodal spectroscopy (MMS) can provide information not provided by each modality. The whole (MMS) is also greater than the sum of the various individual modalities, because the biochemical and morphologic information provided is complementary—that is—the information provided by one technique often answers a question raised by the results of another. For example, for vulnerable atherosclerotic plaque, Raman spectroscopy provides information about the aggregate spectral contribution of foam cells and necrotic core, but raises questions about their individual contributions. Both DRS and light scattering answer those questions by providing specific information about the contribution of foam cells. So by combining the modalities in MMS one can decipher the separate contributions of both foam cells and necrotic core.

Measurements show that for vulnerable plaque, in some cases, two or more modalities are needed to fully characterize the contribution of a single tissue component. For example, as discussed above, oxy- and deoxy-hemoglobin are metabolites that may be key to the spectroscopic diagnosis of cancer. Hemoglobin is a strong tissue absorber and, therefore, it is a potential cause of distortion of tissue fluorescence signatures. This problem has been addressed in part by the use of TMS to derive undistorted IFS signatures. However, measurements in surgical breast biopsies have shown that in extremely bloody operative fields it is not be possible to account for all the absorbance effects of hemoglobin and achieve accurate diagnosis using TMS. On the other hand, hemoglobin is a weak Raman scatterer at NIR excitation wavelengths, and excellent model fits can be achieved for spectra acquired in bloody fields/tissues.

The combination of TMS and Raman spectroscopy in MMS provides a more complete and complementary biochemical, morphologic and metabolic tissue profiles than either TMS or Raman spectroscopy alone resulting in better diagnostic accuracy. Another key advantage in combining both techniques is the potential for depth sensing. TMS and Raman spectroscopy can use different excitation wavelengths, and therefore sample different tissue volumes because of wavelength-dependent differences in absorption and scattering. A Raman source preferably emits in a range of 750 nm to 1000 nm while the fluorescence source can employ one or more laser sources or a filtered white light source. Reflectance measurements preferably use a broadband source such as xenon flash lamp.

This difference in sampling volume can be exploited to provide information about the depth (or thickness) or certain tissue structures of layers. For example, the thickness of the fibrous cap is used to the diagnosis of vulnerable atherosclerotic plaque. The fibrous cap is composed largely of collagen. IFS and Raman spectroscopy both provide information about the contribution of collagen to tissue spectra. Comparison of the results from these two techniques, which use different excitation wavelengths and sample different tissue volumes, may provide information about the thickness of the fibrous cap. DRS and Raman spectroscopy both provide information about the contribution of deoxy-hemoglobin to the tissue spectra. Comparison of the results of these two techniques, which again use different excitation wavelengths and sample different tissue volumes, can provide depth-sensitive information useful in mapping cancers and pre-cancers of breast tissue.

Multimodal spectroscopy (MMS) is a system for spectral diagnosis and efficacy of combining spectroscopic results from TMS and Raman spectroscopy to provide better diagnostic detail and a more comprehensive picture of the biochemical, morphological and metabolic changes that occur in diseased tissues. The probe used in such measurements can be an endoscope or a small diameter probe for insertion through an endoscope channel or a small diameter catheter for insertion in the arterial system, for example.

The Raman methods for the diagnosis of breast cancer are based on a linear combination model similar to that used for peripheral arteries, that yields fit coefficients for epithelial cell nuclei and cell cytoplasm, fat cells, stromal collagen fibers, β-carotene, calcium oxalate and hydroxyapatite and cholesterol-like deposits (corresponding to tissue necrosis). The diagnostic procedure makes use of fit coefficients collagen and fat, two components of the tumor stroma.

Breast cancer, like most cancers, is characterized by abnormal cell proliferation and differentiation as well as increased cell metabolism. Fluorescence, reflectance and LSS provide information about cell metabolism and tissue scatterers such as cell nuclei that is not provided by Raman spectroscopy. Therefore, by combining Raman spectroscopy with fluorescence, reflectance and/or LSS, a method for the diagnosis of breast cancer incorporates contributions from both the malignant epithelial cells and the stroma.

### BRIEF DESCRIPTION OF THE DRAWINGS

FIG. 1A is a schematic illustration of an MMS system in accordance with a preferred embodiment of the invention;

FIG. 1B is a scatter plot of Raman data;

FIG. 2a-2b are basis spectra;

FIGS. 3a-3c are scatter plots of an MMS system;

FIGS. 4a-4c are plots of an MMS system;

FIGS. 5A and 5B shows Raman basis spectra;

FIG. 6a-6c show spectra and fits for MMS modes;

FIG. 7 is a bar graph for hemoglobin concentration;

FIG. 8 shows scattering parameter A for DRS;

FIG. 9 is a plot of the coefficients ratio for IFS;

FIG. 10 is a plot of the Raman parameter for artery samples;

FIG. 11A are graphs of coefficients for artery tissue;

FIGS. 11B-D are Raman, reflectance and fluorescence data of an artery;

FIG. 12 shows sampling depths;

FIGS. 13A and 13B include side and end views, respectively, of a Raman Probe;

FIG. 13C is a side cross-sectional view of a side looking probe;

FIG. 13D is an end view of an MMS probe in accordance with the invention;

FIG. 13E is a forward looking MMS probe with a ball lens;

FIG. 13F is a forward looking MMS probe with a half ball lens;

FIG. 13G graphically illustrates a distal filter system for an MMS probe.

FIG. 14A is a schematic of an MMS system; and

FIG. 14B is another embodiment of an MMS system.

### DETAILED DESCRIPTION OF THE INVENTION

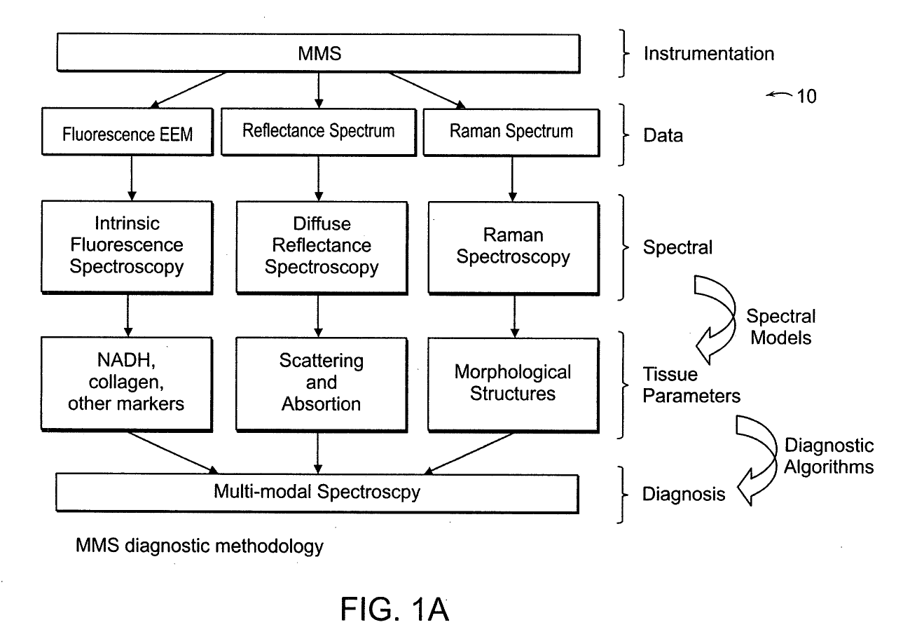

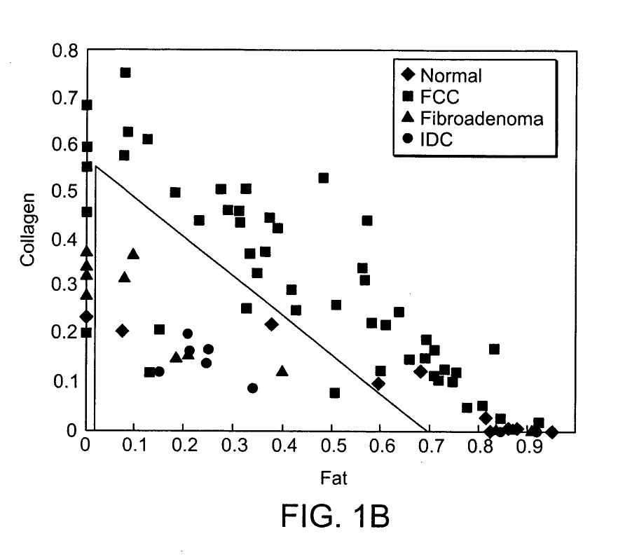

An MMS system is generally illustrated in FIG. 1A. MMS measurements have been performed on surgical biopsies within 30 minutes of surgical resection. Most of the 30 minute time delay was due to inking and sectioning of the specimen performed as part of the routine pathology consultation performed on these specimens for more information on intra-operative margin assessment in breast cancer patients. IFS, diffuse reflectance and Raman spectra were obtained from a total of 223 spectra from 105 breast tissues from 25 patients. Specimens from patients with pre-operative chemotherapy or who underwent re-excisional biopsy were excluded. DRS and IFS spectra were collected using the FastEEM instrument, followed by collection of Raman spectra with a Raman instrument. Care was taken in placing the Raman probe at the same site on the tissue as the FastEEM probe. Once the spectra were acquired, the exact spot of probe placement was marked with colloidal ink for registration with histopathology. The breast specimens were then fixed and submitted for routine pathology examination, which was performed by an pathologist blinded to the spectroscopy results. The histopathology diagnoses were: 32 normal; 55 fibrocystic change, 9 fibroadenoma and 9 invasive carcinoma (all infiltrating ductal carcinoma).

The sampled tissue volume for Raman spectroscopy is 1 mm³. Using the combined biochemical and morphologic spectral model, the data are fit to a linear combination of Raman basis spectra for eight breast tissue components: cell cytoplasm, cell nucleus, stromal collagen fibers, fat cells, β-carotene, collagen, calcium hydroxyapatite, calcium oxalate dehydrate, and cholesterol-like lipid deposits (foci of necrosis). The data were then analyzed prospectively using the fit coefficients for stromal collagen (collagen) and fat cells (Fat) in our Raman algorithm for breast cancer diagnosis. A scatter plot and decision lines for the Raman diagnostic algorithm are shown in FIG. 1B. A comparison of the Raman spectral diagnoses and histopathology diagnoses is shown in Table 1. The Raman algorithm remained quite robust when applied in a prospective manner to these breast specimens, with an overall accuracy of 83%. However, five cases of fibroadenoma were misdiagnosed by Raman as invasive cancer and 4 cases of fibrocystic change were misdiagnosed as cancer.

TABLE 1
Comparison of pathologic diagnosis with that of the Raman diagnostic
algorithm for ex-vivo specimens. The TMS diagnostic algorithm
resulted in an overall accuracy of 81% (85/105).

| | Pathology | | | |
|---|---|---|---|---|
| | | Fibro- | | |
| | | cystic | Fibro- | Invasive |
| | Normal | Change | adenoma | Carcinoma |
| | (32 sam- | (55 sam- | (9 sam- | (9 sam- |
| Raman | ples) | ples) | ples) | ples) |
| Normal | 30 | 7 | 0 | 0 |
| Fibrocystic Change | 2 | 41 | 0 | 0 |
| Fibroadenoma | 0 | 3 | 4 | 1 |
| Invasive Carcinoma | 0 | 4 | 5 | 8 |

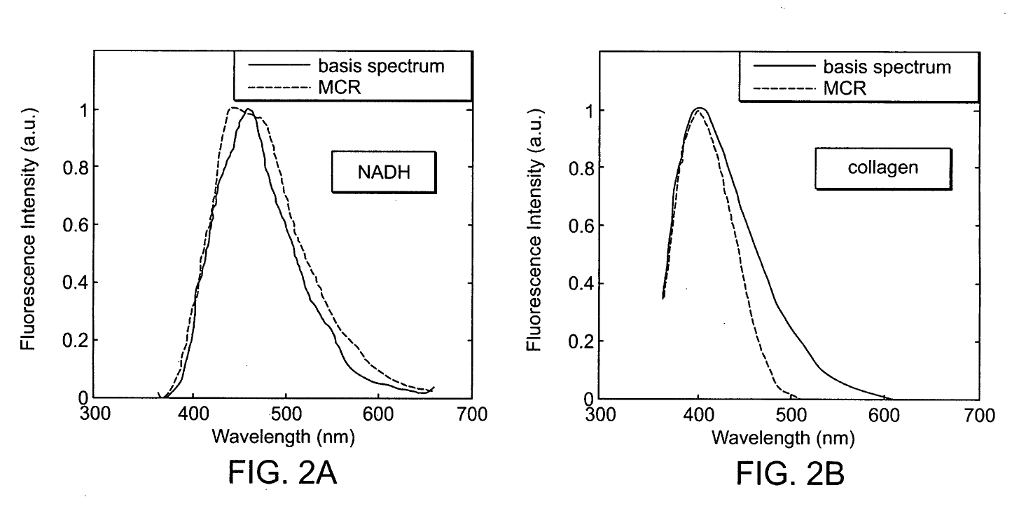

IFS were extracted from the combined fluorescence and DRS. The IFS spectra were analyzed using multivariate curve resolution (MCR) with non-negativity constraints, a standard chemometric method, to extract two spectral components at each excitation wavelength. The resulting MCR-generated spectral components at 340 nm are shown in FIG. 2a and FIG. 2b, and represent NADH and collagen, respectively, because they are similar to their measured IFS spectra. The spectra are similar, but not identical, as both the lineshape and wavelength maximum of a fluorescence peak obtained from a solution of a pure component is known to be different than that obtained from the same component in a different chemical environment, such as tissue.

For diffusive scattering (μ^{s}′), wavelength dependence of the form Aλ^{-B} is used. Two absorbers, oxyhemoglobin and β-carotene, were used to model the extracted absorption coefficient μa. Therefore, DRS provided, among other parameters, the amplitude of the scattering coefficient, A, and the concentration of oxyhemoglobin.

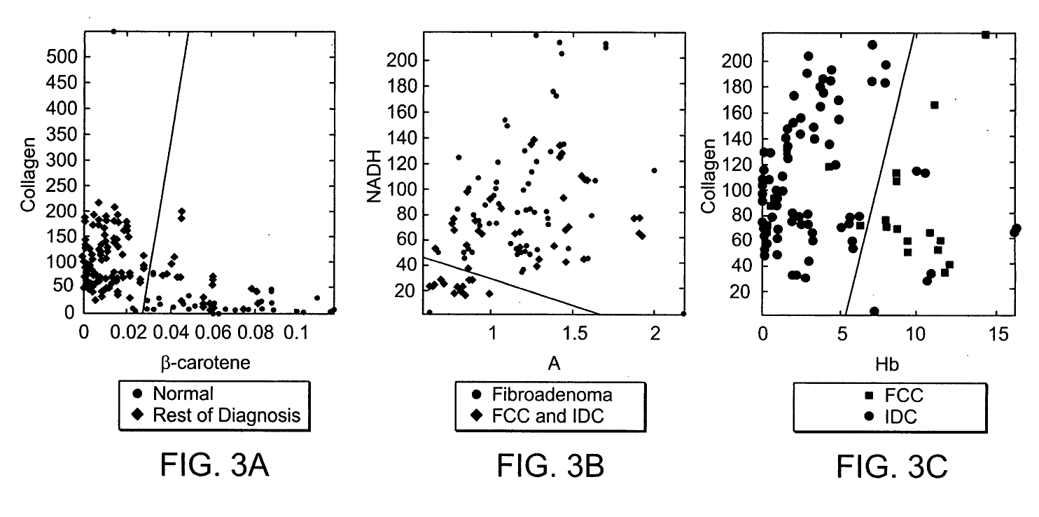

The TMS diagnostic method used logistic regression and leave-one-out cross validation, and the analysis was performed in sequential fashion. Scatter plots and decision lines for each step of the diagnostic method are shown in FIG. 3a-3c. Normal tissue was identified using the Raman fit coefficients for both collagen and β-carotene (FIG. 3a). The finding of low fit coefficients for collagen and β-carotene correlates with histopathology, as normal breast tissue consists largely of adipose tissue, the fat cells which contain large amount of lipid-soluble β-carotene. After the normal tissue was excluded, fibroadenoma was discriminated from fibrocystic change and invasive breast cancer, using the DRS scattering parameter A and IFS NADH fit coefficients (FIG. 3b). Fibrocystic change was distinguished from invasive breast cancer using the DRS oxyhemoglobin and IFS collagen fit coefficients at 340 nm (FIG. 3c). This diagnostic method uses contributions from both the cells (NADH) and the stroma (collagen). However, it is unclear why the fit coefficient for collagen and scattering parameter A should be lower for fibroadenoma than for invasive carcinoma and fibrocystic disease, or the fit coefficients for oxyhemoglobin should be higher for invasive breast cancer than for fibrocystic disease. A comparison of the TMS spectral diagnoses and histopathology diagnoses is shown in Table 2. The overall accuracy (correct prediction of each of the pathologies) is 87.6% (92/105). Although the overall accuracy of the two techniques is comparable in this small data set, all of the invasive carcinomas were diagnosed correctly by TMS and only 4 normals or fibrocystic changes were misclassified as invasive carcinoma.

TABLE 2
Comparison of TMS and histopathologic diagnosis for ex vivo
study of fresh surgical breast biopsies. The TMS diagnostic
algorithm had an overall accuracy of 87.6% (92/105).

| | Pathology | | | |
|---|---|---|---|---|
| | | Fibro- | | |
| | | cystic | Fibro- | Invasive |
| | Normal | Change | adenoma | Carcinoma |
| | (32 sam- | (55 sam- | (9 sam- | (9 sam- |
| TMS | ples) | ples) | ples) | ples) |
| Normal | 27 | 7 | 0 | 0 |
| Fibrocystic Change | 2 | 47 | 0 | 0 |
| Fibroadenoma | 0 | 0 | 9 | 0 |
| Invasive Carcinoma | 3 | 1 | 0 | 9 |

The measurements were obtained using TMS and Raman spectroscopic techniques independently can also be obtained using a combined diagnostic procedure. In developing the MMS algorithm, only parameters that were diagnostic in one of the three individual spectroscopic modalities were used. The diagnostic parameters from TMS are scattering parameter A, and the fit coefficient for oxyhemoglobin, β-carotene, and NADH and collagen by IFS at 340 nm excitation wavelength. The diagnostic Raman parameters are the fit coefficients for fat and collagen. Like the TMS diagnostic procedure, this algorithm incorporates contributions from both the epithelial cells (NADH) and stroma (collagen).

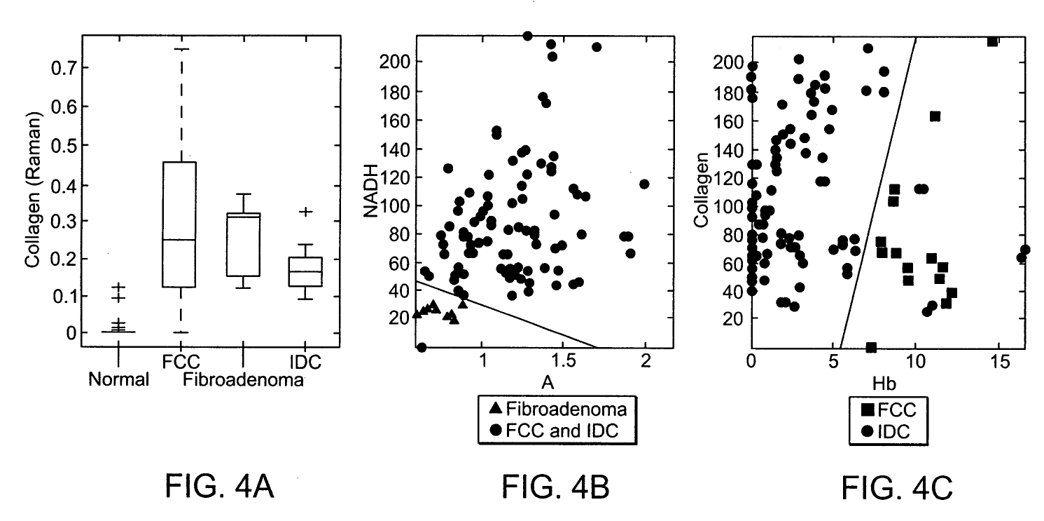

The MMS diagnostic method was developed using logistic regression and leave-one-out cross validation. As with TMS, the analysis is performed in sequential fashion. FIGS. 4a-4c displays the scatter plots and decision lines for each of the three steps performed in the MMS diagnostic algorithm. First, normal tissue was identified using the Raman fit coefficient for collagen. This is the only change in this algorithm than that used for TMS, where the first step was identification of normal tissues using the intrinsic fluorescence fit coefficient for collagen at 340 nm (FIG. 4a). The next two steps are identical to those in the TMS diagnostic algorithm, with fibroadenoma distinguished from fibrocystic change and invasive carcinoma using scattering parameter A and the fit coefficient for NADH (FIG. 4b), and fibrocystic disease distinguished from invasive breast cancer using the fit coefficients for oxy-hemoglobin (FIG. 4c). A comparison of the MMS spectral diagnoses and histopathology diagnoses is shown in Table 3. The overall accuracy is 92% (92/105), and is only slightly improved for MMS as compared to TMS. As with TMS, all 9 invasive carcinomas were diagnosed correctly by MMS. But this time, only 2 fibrocystic changes and no fibroadenoma are diagnosed as invasive carcinoma.

TABLE 3
Comparison of MMS and histopathologic diagnosis for the
ex vivo study of surgical breast biopsies. The MMS diagnostic
algorithm had an overall accuracy of 92.4%.

| | Pathology | | | |
|---|---|---|---|---|
| | | Fibro- | | |
| | | cystic | Fibro- | Invasive |
| | Normal | Change | adenoma | Carcinoma |
| | (32 sam- | (55 sam- | (9 sam- | (9 sam- |
| Multimodal | ples) | ples) | ples) | ples) |
| Normal | 30 | 4 | 0 | 0 |
| Fibrocystic Change | 2 | 49 | 0 | 0 |
| Fibroadenoma | 0 | 0 | 9 | 0 |
| Invasive Carcinoma | 0 | 2 | 0 | 9 |

Table 4 shows a detailed comparison of the diagnostic performance of all three methods, Raman, TMS and MMS, with MMS providing the best sensitivity and specificity, as well as overall accuracy. By introducing a parameter from the Raman model to the first step a greater number of correctly diagnosed normal tissues. FIG. 4a is a box plot, which illustrates the average values (red line), the interquartile range (blue box), and outliers (red plusses), of collagen content for each pathology. Previously, the collagen content from TMS was analyzed in this manner but did not show the same success. Although both Raman and TMS (and thus MMS) are sensitive to collagen, each uses a different wavelength of light (Raman at 830 nm and TMS at 340 nm). Therefore, their sampling volumes are different. This fact explains why collagen fit coefficients extracted via Raman spectroscopy do not strongly correlate with collagen fit coefficients extracted using TMS. This is likely because of the different sample volumes (depths) of the TMS and Raman modalities. With a smaller sampling volume, TMS did appear to sample deep enough into the tissue to assess collagen adequately.

The results indicate that MMS, a combination of DRS, IFS, and Raman spectroscopy provides better results than those obtained from each technique alone. This can result from the combined MMS diagnostic algorithm combines spectral parameters derived from both epithelial cells and stroma and (taken together) have a larger sample volume.

TABLE 4
Comparison of performance of Raman, TMS and MMS
algorithms for the diagnosis of breast cancer.

| | Modality | | |
|---|---|---|---|
| Performance | Raman | TMS | MMS |
| Sensitivity | 89% | 100% | 100% |
| Specificity | 91% | 96% | 98% |
| Overall Accuracy | 81% | 88% | 92% |

As in breast cancer, the development of atherosclerosis is governed by subtle chemical and morphological changes in the arterial wall, manifesting themselves in the development of a plaque that causes luminal obstruction. Many of these changes are the result of metabolically active inflammatory and smooth muscle cells, such as foam cells, that degrade LDL and release it into the necrotic core in the form of ceroid and other LDL degradation byproducts.

The preferred method for the diagnosis of atherosclerosis are based on a linear combination model that yields fit coefficients for 10 morphological components of artery wall, including collagen fibers (CF), elastic lamina (EL), smooth muscle cells (SMC), adventitial adipocytes (AA), cholesterol crystals (CC), β-carotene crystals (β-CC), foam cells/necrotic core (FC/NC) and calcium mineralizations (CM). A preferred algorithm was developed for classification of lesions as non-atherosclerotic, non-calcified plaque and calcified plaque. This diagnostic algorithm was based on combined fit coefficients for cholesterol crystals+foam cells/necrotic core (the latter two having indistinguishable Raman basis spectra) and the fit coefficient for calcium mineralizations.

A preferred embodiment relates to a procedure for measuring vulnerable plaque. These are most often plaques with a thin fibrous cap overlying a large necrotic lipid core, and may have other features of vulnerability including foam cells and other inflammatory cells, intraplaque hemorrhage or thrombosis. A second Raman algorithm capable of diagnosing vulnerable plaque with about the same sensitivity and specificity as a previous algorithm for plaque classification (˜85-95%). This second algorithm for the diagnosis of vulnerable plaque makes use of the fit coefficients of 5 artery morphological components: the combined fit coefficients for foam cells+necrotic core and the fit coefficient for calcifications, as in the previous algorithm, plus the fit coefficients for collagen and hemoglobin. A preferred algorithm for the diagnosis of vulnerable plaque involves using spectral parameters that distinguish between metabolically active foam cells and the non-metabolically active necrotic core.

Fluorescence, reflectance and LSS provide information about cell metabolism and tissue scatterers such as foam cells, the cytoplasm of which is filled with a foam-like aggregate of lipid-filled lysosomal vesicles where the metabolism and degradation of LDL takes place. Therefore, by combining Raman spectroscopy with fluorescence, reflectance and optionally LSS, an algorithm for the diagnosis of vulnerable plaque incorporates contributions from metabolically active, potential scatterers like foam cells as well as non-metabolically active plaque constituents like the necrotic core. But, MMS has a further advantage for the diagnosis of vulnerable plaque, and that is the ability to provide depth information about key biochemical and morphologic structures like the fibrous cap, that too undergoes degradation, this time, by matrix metalloproteinase that renders it more prone to rupture.

In vitro measurements of MMS for the diagnosis of vulnerable plaque using 17 frozen archival tissues from carotid endarterectomies have been performed.

TMS spectra were collected using the FastEEM instrument and Raman using the clinical Raman system, with the associated probes. Care was taken in placing the Raman probe at the same site on the tissue as the FastEEM probe. Once the spectra were acquired, the exact spot of probe placement was marked with colloidal ink for registration with histopathology. The artery specimens were then fixed and submitted for routine pathology examination, which was performed by a cardiovascular pathologist blinded to the spectroscopy results. The histopathology examination of the lesions included an assessment of a number of histologic features of vulnerable plaque, including thickness of the fibrous cap, size of the necrotic core, superficial foam cells, intraplaque hemorrhage and ulceration. The histopathology results are summarized in Table 5. A vulnerable plaque index (VPI) was assigned to each specimen. Of the 17 lesions, 4 exhibited VPI scores ≧10 and were classified as vulnerable plaques.

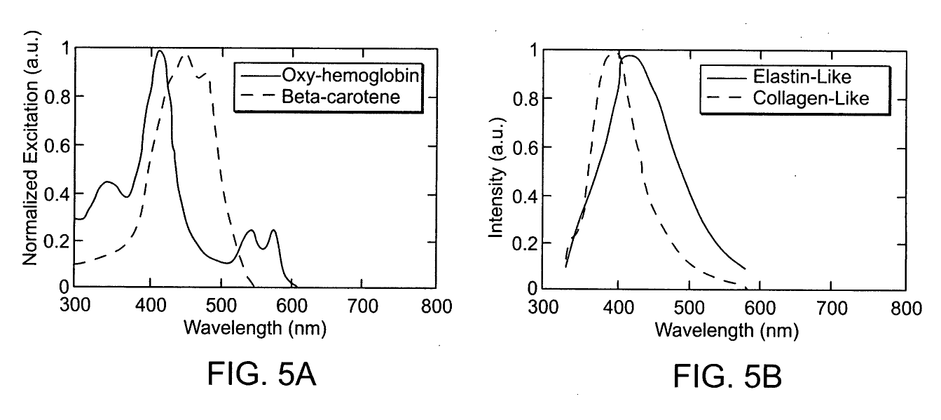

MMS spectral analysis for artery was similar to that for the breast. Again, OLS is used to fit the Raman data using the morphological model. The DRS spectra were fit using the diffusion theory model. Modeling of the DRS spectra yielded, among other parameters, scattering coefficient A and hemoglobin concentration. IFS were analyzed using MCR with non-negativity constraints to find two spectral components at 308 nm and 340 nm. The IFS data was fit using ordinary least squares (OLS) using the two MCR components as the model. The Raman basis spectra, DRS extinctions and IFS MCR components are shown in FIGS. 5A and 5B.

TABLE 5
Morphological characteristics of the 17 specimens.

| | | | Intimal or | Necrotic | | | | |
|---|---|---|---|---|---|---|---|---|
| | | | Fibrous cap | Core | Foam Cell | Foam Cell | | |
| | SNOMed | | Thickness | Thickness | Depth | Grade | Intraplaque | |
| | Class. | VPI | (microns) | (microns) | (microns) | (0−3+) | Hemorrhage | Ulceration |
| 1 | IF | 5 | 24-64 | NA | NA | NA | NA | NA |
| 2 | IF | 5 | 40-80 | NA | NA | NA | NA | NA |
| 3 | ATS | 0 | 480-500 | NA | 480 | 3+ | NA | NA |
| 4 | ATS | 5 | 240-440 | NA | 40 | 1+ | NA | NA |
| 5 | ATS | 0 | 456-536 | NA | 456 | 2+ | NA | NA |
| 6 | ATM | 5 | 200-320 | 400 | 280 | 2+ | NA | NA |
| 7 | ATM | 5 | 460-640 | 560 | NA | NA | NA | NA |
| 8 | ATM | 5 | 440-500 | 4800 | 440 | 2+ | NA | NA |
| 9 | ATM | 5 | 1000-1500 | 6400 | 1800 | 1+ | NA | NA |
| 10 | ATM | 5 | 520-640 | 1340 | 640 | 2+ | NA | NA |
| 11 | CATM | 5 | 140-160 | 1840 | 68 | 1+ | NA | NA |
| 12 | CATM | 7 | 120-480 | 4000 | 120 | 1+ | NA | NA |
| 13 | CATM | 5 | 1440-1600 | 240 | 256 | 1+ | NA | NA |

IF = infimal fibroplasias, ATS = atherosclerotic, ATM = atheromatous, FS = fibrotic-sclerotic, C = calcified.

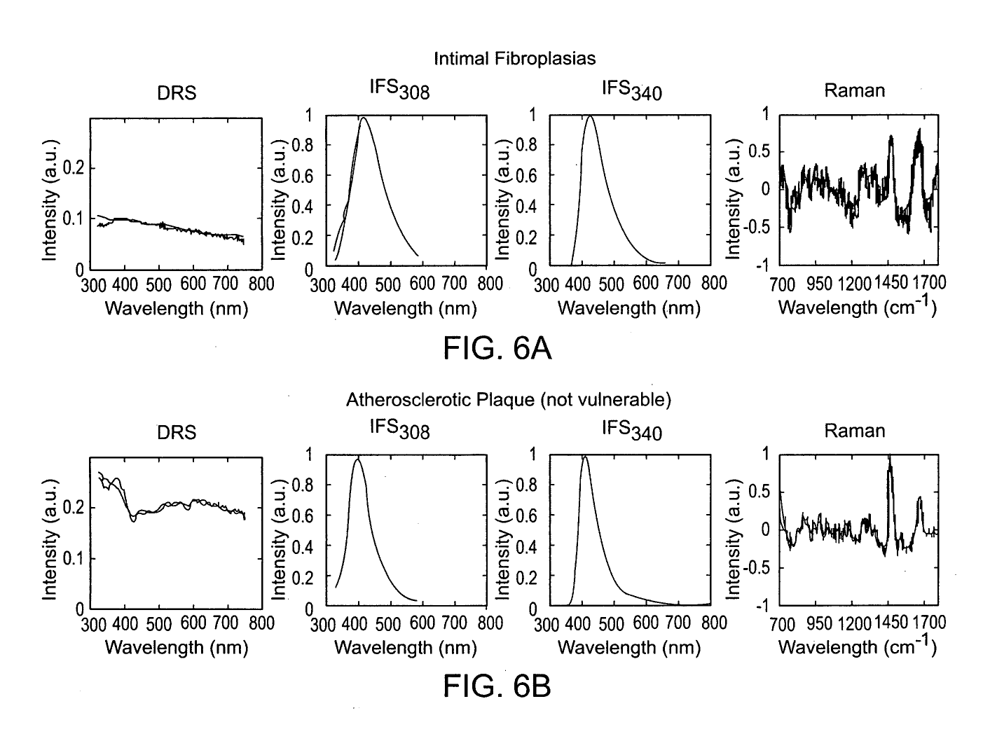

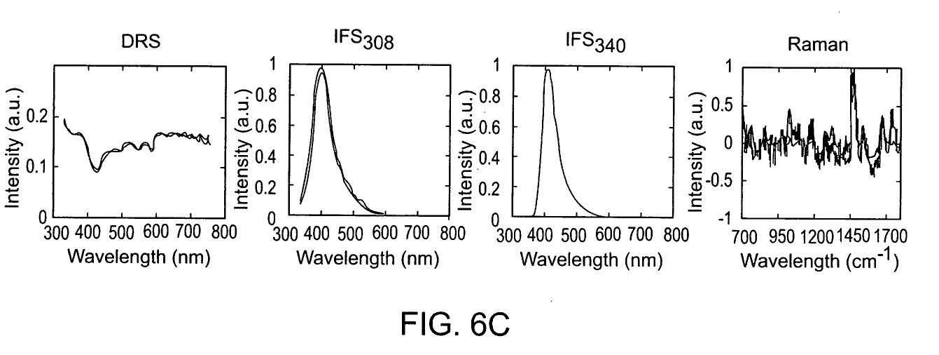

FIG. 6a-6c shows the spectroscopic data and model fits for three different artery lesions, an intimal fibroplasia (a), a non-vulnerable plaque (b) and a vulnerable plaque (c). All of the MMS spectra could be fit very well using the previously described models.

Four spectral parameters were correlated with the histopathologic features of vulnerable plaque: DRS scattering parameter A and hemoglobin concentration; an IFS parameter ρ=C₃₀₈/C₃₄₀, where C₃₀₈ and C₃₄₀ are the contributions of the more blue-shifted MCR basis spectra; and the Raman parameter Σ=CC+FC/NC, where CC and FC/NC are the relative contributions in the Raman spectra of cholesterol crystals and foam cells+necrotic core, respectively. The diagnostic potential as it relates to assessing plaque vulnerability for each of the spectral parameters will be discussed separately in the next paragraphs.

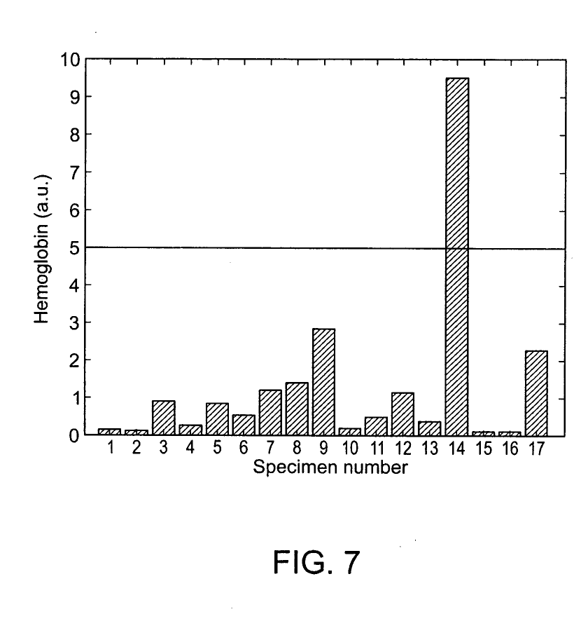

As described earlier, intraplaque hemorrhage is a marker of plaque vulnerability. Histopathology indicates that the lesion in specimen #14 is the site of acute intraplaque hemorrhage (Table 5); whereas the other lesions not hemorrhagic. FIG. 7 displays the hemoglobin concentration fit parameters of the 17 specimens obtained from the DRS spectra. The lesion in specimen #14 exhibits a distinctly high c_{Hb}, and a threshold value of c_{Hb}=5 separates it from the remaining lesions. This suggests that the concentration of hemoglobin inside the arterial wall, measured with DRS to sense acute intraplaque hemorrhage.

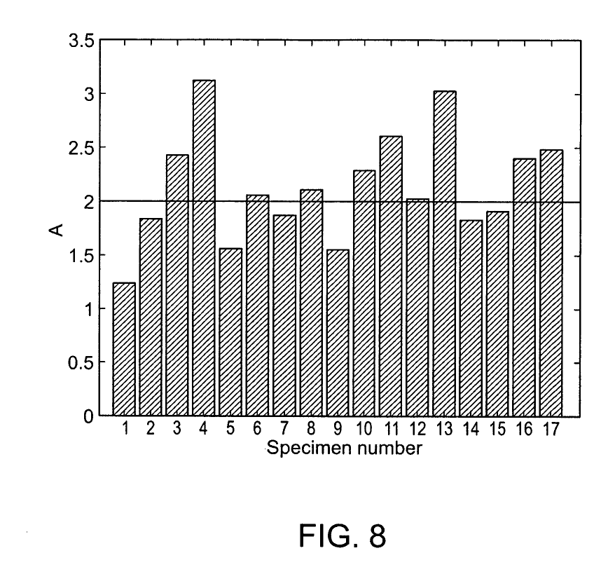

Superficial foam cells are important in assessing plaque vulnerability as they are often present in the fibrous cap near plaque erosions and ruptures, and are a likely source of MMPs that degrade the fibrous cap and lead to plaque rupture. FIG. 8 displays the DRS scattering parameter A (relative units) for the 17 specimens. Foam cells are present in all 10 lesions with A>2, where they occur at an average depth of 250 microns below the intimal surface of the plaque (Table 5). In contrast, foam cells are observed in only 2 of the 7 lesions with A<2, and these foam cells tend to reside deeper in the plaque, at an average depth of 1100 microns (Table 5). Given the several hundred micron penetration depth of DRS at visible wavelengths, DRS does not sense such deep foam cells, which are not clinically relevant to plaque vulnerability. Hence the scattering parameter A appears to be a measure of the presence of superficial foam cells. The correlation of A with foam superficial suggests that the presence of foam cells near the tissue surface can markedly enhance scattering, and that foam cells, which contain a high concentration of lysosomal vesicles, are strong light scatterers. In addition this data suggests that, using parameter A, the method differentiates the presence of foam cells from that of necrotic core, which Raman spectroscopy alone cannot do.

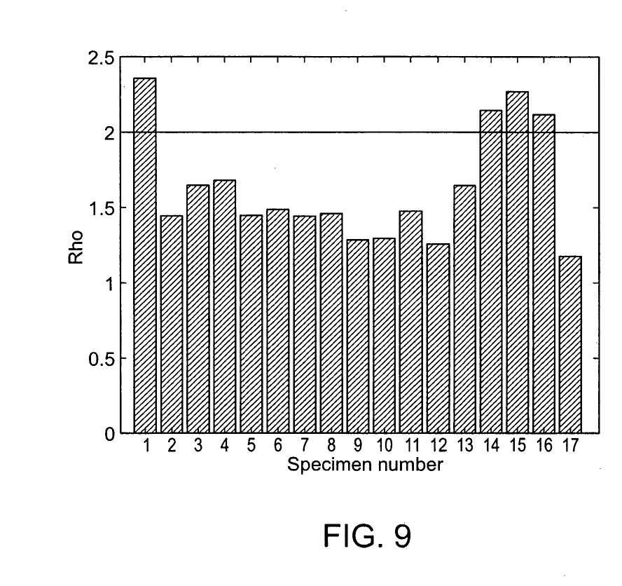

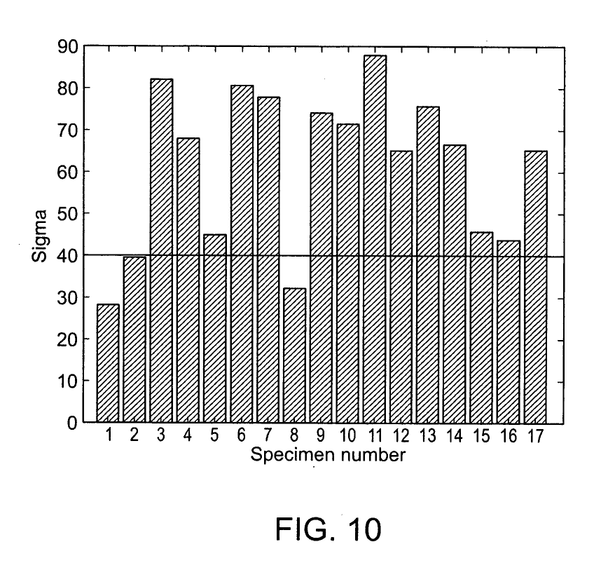

As discussed above, an important feature of vulnerable plaque is the presence of a thin fibrous cap. A cap thickness of less than 65 μm is an established criterion of vulnerability. IFS spectra at 308 and 340 nm excitation wavelengths were obtained to parameterize fibrous cap thickness. Two MCR spectral components to be associated with collagen and/or elastin, structural proteins that characterize the upper layers of both normal artery (normal intima) and atherosclerotic lesions (fibrous cap). Comparing the MCR spectra to the known spectral of those fluorophores, the red-shifted spectrum resembled elastin while the blue-shifted spectrum is similar to collagen (FIG. 5). The corresponding fit coefficients, C₃₄₀ and C₃₀₈, relate to the amount of collagen present within the tissue volume sampled. The sampling depth with 340 nm excitation (˜60 μm) is greater than that with 308 nm excitation (˜50 μm). Thus, C₃₄₀ provides information about collagen and elastin distributed over a much greater depth compared to that provided by C₃₀₈. Hence, the ratio ρ=C₃₀₈/C₃₄₀ can provide information about the thickness of the fibrous cap. FIG. 9 plots ρ for the 17 specimens. Lesions with the highest values (ρ>2, specimens #1 and 14-16) have the lowest average intimal or fibrous cap thicknesses, all below 50 μm. Conversely, for each of the remaining specimens, for which ρ<2, the average cap thickness is greater than 50 μm. The one exception to this is Specimen #17, an ulcerated plaque, which has a variable fibrous cap thickness, ranging from 0 to 200 μm, and yet it has a ρ<2. Nevertheless, these results indicate that a threshold value ρ=2 can be used to identify thin fibrous caps. For Raman spectroscopy, the parameter Σ=CC+FC/NC is an indicator of the presence of necrotic material, foam cells and cholecterol crystals. The values of Σ for the 17 carotid artery specimens are plotted in FIG. 10. Specimens rich in foam cells or necrotic core exhibit larger values of Σ. A threshold value of Σ=40 separates specimens of low and high overall lipid content. The only exceptions are specimens #14 and #15, which have high values of Σ although histopathology indicates the absence of foam cells and/or necrotic core. These two specimens are fibrotic-sclerotic plaques. They are morphologically unusual, demonstrating a well-developed fibrous cap but lacking an extracellular necrotic core and cholesterol crystals. These can be viewed as end stage plaques.

The key spectroscopic parameters obtained from IFS, DRS and Raman spectroscopies are displayed together in Table 6 for all 17 specimens. This method uses yes/no results based on the threshold values rather than numerical values. Each column represents a spectroscopic marker of a histologic feature of vulnerable plaque: Hb, indicative of intraplaque hemorrhage; scattering parameter A, indicative of foam cells close to the surface; ρ, indicative of fibrous cap thickness; and Σ, indicative of the build up of necrotic core material. Note that 3 of the 4 vulnerable plaques can be identified by detecting a thin cap (ρ>2) together with another parameter such as A or Σ.

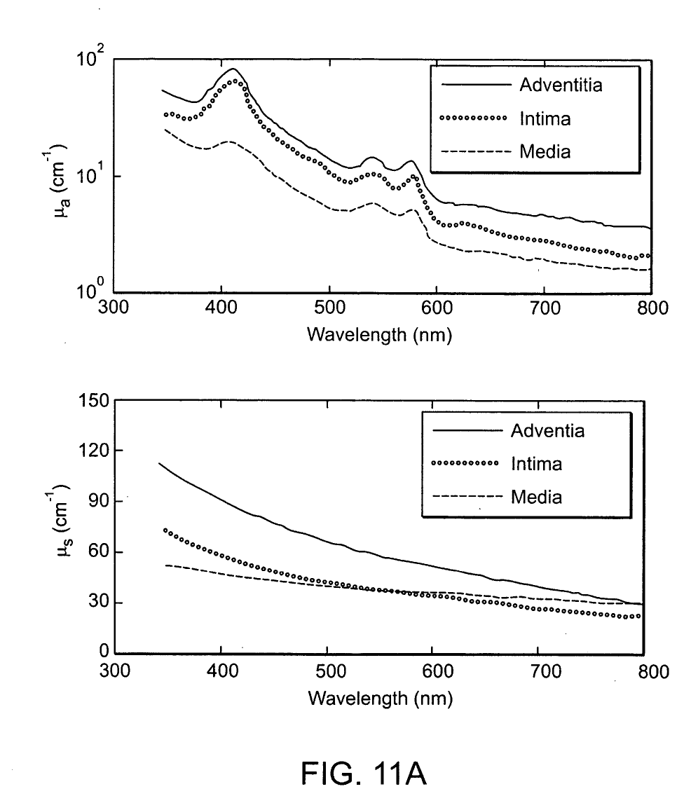

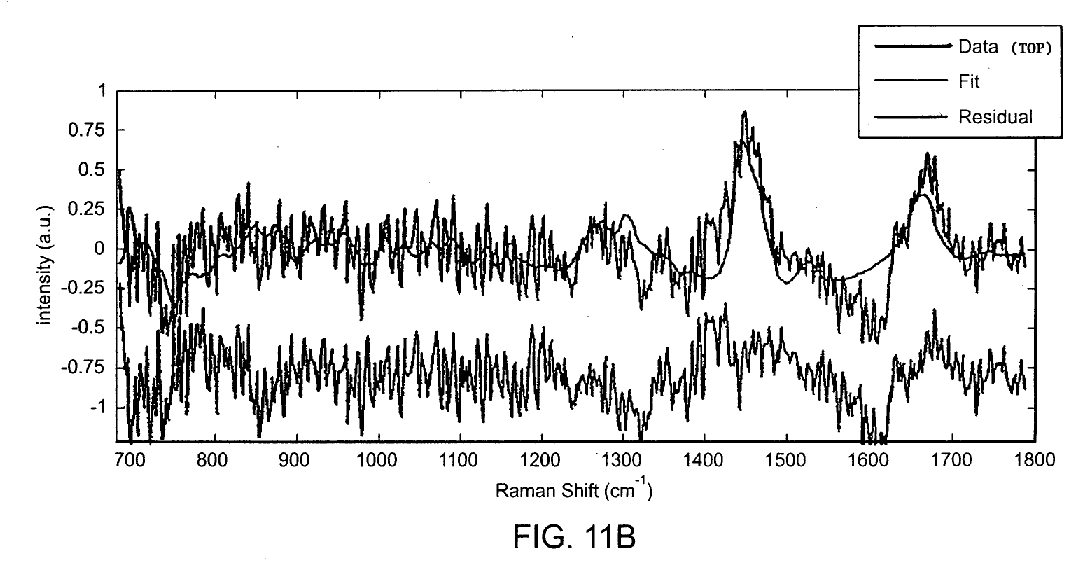

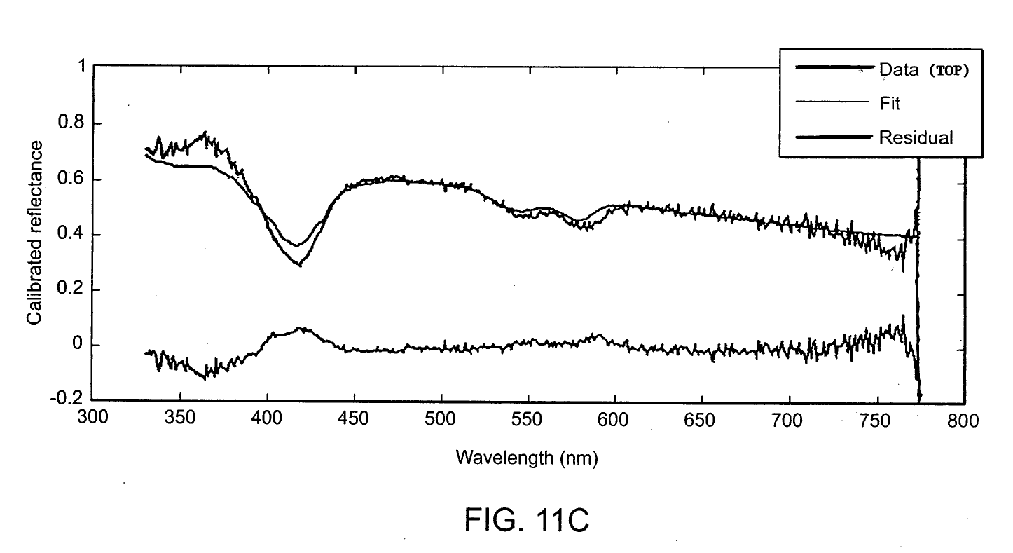

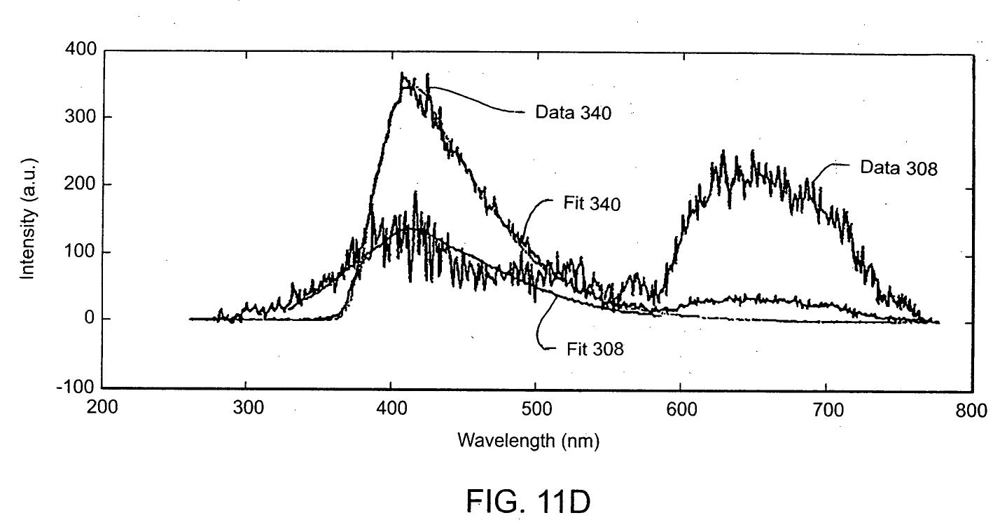

The ability of MMS to provide depth-sensitive information is more relevant to measurements of atherosclerosis than those of breast cancer because of the layered structure of the arterial wall. Define the optical penetration depth as the depth at which the power of light incident on a tissue sample falls to 1/e of its incident value. Generally the optical properties of aorta indicates penetration depths of about 90, 150 and 1200 microns for light of wavelengths 308, 340 and 830 nm, respectively. The penetration depths at different IFS wavelengths were measured by incrementally stacking 20 μm thick sections of aortic media. The FastEEM probe tip was placed in contact with the tissue and the transmitted power was measured as a function of tissue thickness. The penetration depths at 308 and 340 nm were measured as 85 and 105 μm, respectively. These values correspond with prior results especially noting the variability of human tissue. They also agree with estimates obtained from the formula δ=1/μ_{eff=}1/\sqrt{3μₐ(μₐ+μ′ₛ)}, using the known scattering and absorption properties of arterial tissue at different wavelengths; FIG. 11A gives the μₐ and μₛ′ in the visible wavelength range.

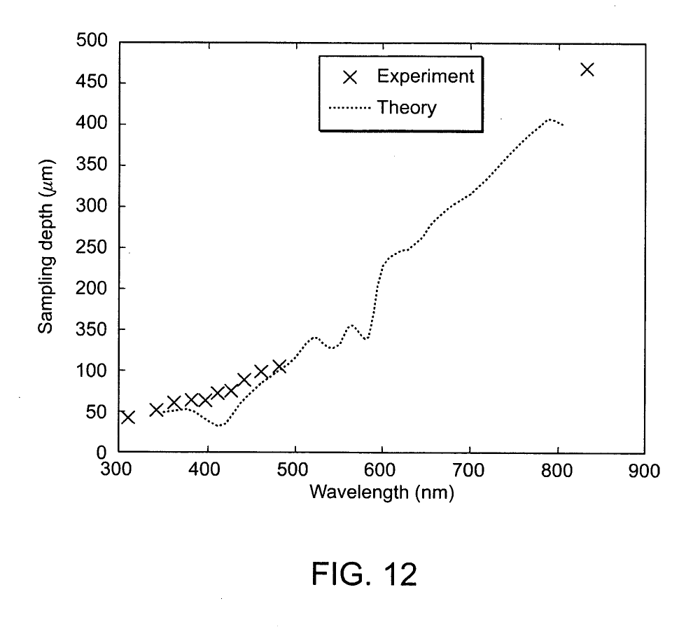

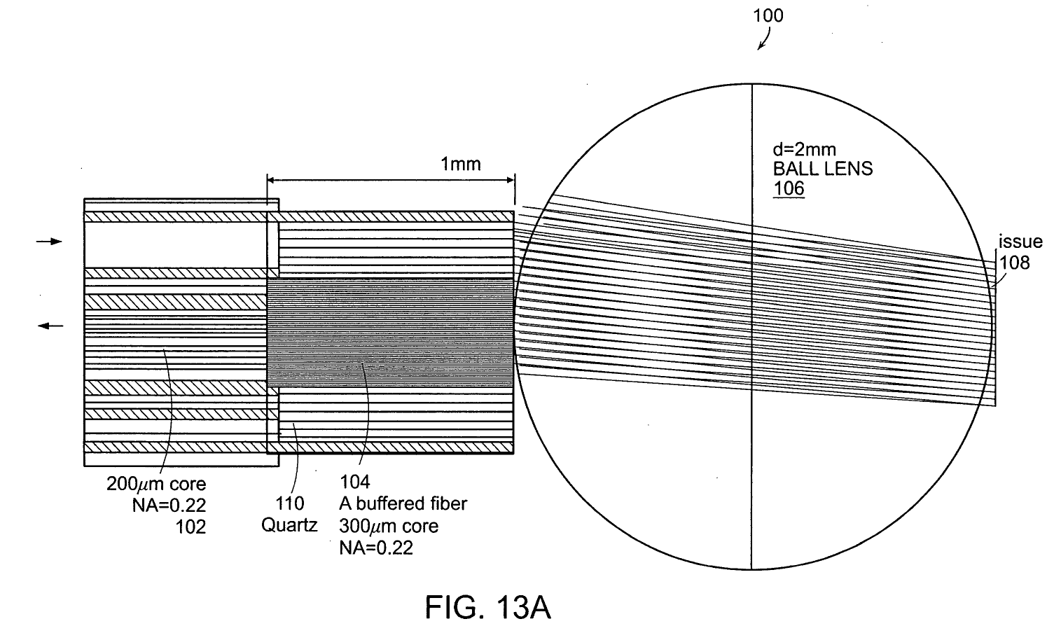

Note that in the single-ended geometry of our artery measurements (i.e. the probe both delivers and collects light at the same position) the sampling depth, which can be defined as 1/δₛ=1/δₑₓ+1/δₑₘ, where δₑₓ and δₑₘ are the penetration depths of the excitation and emission light, respectively. The sampling depth characterizes the attenuation of both the excitation and the emitted light, which can be at a longer wavelength, as in the case of fluorescence or Raman scattering. Thus the sampling depths of IFS₃₀₈ and IFS₃₄₀ are much smaller: 50 and 60 μm, respectively, taking into account the longer wavelengths of the emitted light. A previous measurement established a sampling depth of 470 μm for Raman spectroscopy of artery using 830 nm excitation. In the following, use 50, 60 and 470 um as the sampling depths at 308, 340 and 830 nm, respectively. Note that the definition of penetration as the length where light is attenuated to 1/e of its original value is somewhat arbitrary and that, optionally the device can sample deeper than those values. Similarly, different wavelength regions of the diffuse reflectance spectra sample tissue at different depths. In general, short wavelength IFS (308 nm, in particular) provides information about the top layer (intima/fibrous cap), longer wavelength IFS samples somewhat deeper, and Raman spectroscopy has the greatest sampling depth. FIG. 12 gives the sampling depths at various wavelengths in the range 308-830 nm, comparing values from our experimental studies those calculated from the literature (the emission wavelength is chosen to be the same as the excitation so δₛ=δₑₓ/2).

Multimodal spectroscopy (MMS) is a spectral diagnosis technology that combines spectroscopic results from TMS and Raman spectroscopy to provide more accurate disease diagnosis and a more comprehensive picture of biochemical, morphological and metabolic state of the tissue as it relates to disease pathogenesis and pathophysiology. FIGS. 11B-D illustrate in vivo Raman (FIG. 11B) diffuse reflectance (FIG. 11C) and intrinsic fluorescence (FIG. 11D) spectra taken of a femoral artery. The artifact between 600 and 700 nm in the IFS spectrum is due to the surgical light in the room which can be turned off during use.

The results have demonstrated that combining information from Raman, fluorescence and reflectance spectroscopies provides better diagnostic accuracy than that provided by any one of the spectroscopic techniques independently, and that differences in sampling volumes can be used to advantage for depth sensing.

The present invention relates spectroscopic diagnosis of a wide range of diseases including oral, esophageal, colon and cervical cancer, as well as the diagnosis of vulnerable atherosclerotic plaque and breast cancer. A preferred embodiment spectroscopically extracts biochemical, morphologic and metabolic information related to features of plaque vulnerability or predictive of breast cancer. More than rendering precise disease diagnoses, the system extracts accurate biochemical, morphologic and metabolic information about tissue composition. The system stores IFS morphological basis spectra using microspectroscopy, and performs ex vivo and in vivo tissue measurements using DRS, IFS, and Raman spectroscopic techniques.

Combined MMS spectral data provides insight into depth dependent morphological features of breast cancer (collagen) and vulnerable plaque (fibrous cap thickness and superficiality of foam cells). These measurements simultaneously collect and analyze Raman, DRS and IFS spectra from the same spot without registration errors using an MMS probe.

Quantitative information about biochemical and morphological tissue components are provided from DRS and Raman spectra using basis spectra in our linear combination model. IFS can also provide quantitative information. Meaningful data modeling can be obtained using fluorescence basis spectra measured from biochemical and morphologic tissues structures measured in situ uses the IFS technique to remove the artifacts of tissue absorption and scattering. This can be useful as basis spectra obtained by microspectroscopy of thin (<6 μm) tissue sections or cell cultures can have little or no scattering or absorption effects, and thus may not model uncorrected raw fluorescence spectra as well as IFS spectra.

To build representative libraries of basis spectra, 50-100 spectra were acquired each from a variety of tissue and cellular sources. Tissue handling and preparation methods can lead to spectral distortions. For example, increased absorption has been observed in frozen-thawed tissue, possibly the result of red blood cell lysis, with a concomitant decrease in tissue fluorescence. These changes are less significant in artery wall than in epithelial tissues. Several steps are taken to minimize these artifacts in the collection of IFS basis spectra. First, all IFS basis spectra are collected from freshly excised tissues within 30-60 minutes of excision.

In the case of artery, basis spectra are obtained initially from cryostat sections of fresh tissue that has been immediately snap frozen in liquid nitrogen. Basis spectra are obtained on these sections within minutes of preparation. The passively thawed frozen sections maintained in a humid chamber to prevent drying.

Optionally, basis spectra obtained either from fresh tissue sections (or short term organ cultures) maintained in a balanced electrolyte solution such as Hanks Balanced Salt solution at neutral pH. Under these conditions it is known that tissue remains viable for at least 90 minutes, with minimal changes in fluorescence. Basis spectra can also be obtained from live human cell cultures, where appropriate, to provide a relatively pure population of cells. Cell cultures from which basis spectra may be obtained for artery studies include primary cultures of normal human endothelial and smooth muscle cells and various cell culture models of foam cells, such as LDL fed human alveolar macrophages. Cell cultures from which basis spectra may be obtained for the breast studies include primary cell cultures of normal breast epithelial cells, myoepithelial cells and fibroblasts and human breast cancer cell lines.

The basis spectra can be collected using a confocal microscope adapted for TMS microspectroscopy. A confocal fluorescence system uses excitation light generated by the FastEEM instrument. The excitation light from the FastEEM is delivered from a 200 um fiber, focused to 100 um aperture and collimated. The collimated light is delivered down to the objective using a neutral density beam splitter (90/10) and collected light from the thin tissue is be focused to a confocal pinhole. This light is delivered to the FastEEM spectrograph and CCD via optical fibers. The microscope stage is programmed to FastEEM scan in the features of interest. A bright field image of the specimen is obtained and used for registration between pathology and spectroscopy. The FastEEM software is synchronized for operation between the microscope and FastEEM excitation source and CCD camera.

With the library of biochemical and morphological basis spectra morphological basis spectra (of such structures as foam cells in atherosclerosis and epithelial cell nuclei and cytoplasm in breast cancer) are fit with the same linear combination method used previously for Raman spectroscopy, using biochemical basis spectra to determine their precise chemical composition and identify the fluorophores characteristic of each structure. The basis spectra are also fit to ex vivo IFS tissue spectra, and quantitative information about the presence of fluorophores (tryptophan, collagen, elastin, NADH, FAD, β-carotene) and the morphologic structures they comprise, is extracted. Using this quantitative spectral information obtained from all three spectral modalities, an automated method to characterize morphological components associated with disease state, including their depth profiles, is provided. Quantization of the biochemical and morphologic composition of the tissues is incorporated into algorithms for the diagnosis of vulnerable plaque and breast cancer. Similar basis spectra libraries, spectral models and diagnostic algorithms are used for cancers of the oral cavity, colon, bladder and cervix.

Using at least 200 spectra each from ex vivo fresh arterial (carotid and femoral) and breast tissues from at least 40 different patients spectra are acquired using the MMS instrument using the integrated MMS probe. The location of the spectroscopic site is established by attaching a metal sleeve to the probe that can make a shallow incision around the site. After removing the probe, the location can be marked with an ink dot. The sample can be fixed in formalin and submitted for histopathological examination, by a pathologist. Both spectral analysis and quantitative image analysis (QIA) of the samples is performed in parallel, using the same tissue site for both measurements.

To evaluate the depth sensing capabilities of different fluorescence excitation wavelengths, Monte Carlo models are employed to simulate light propagation within tissue. Monte Carlo models can have simple layered structures with physiological dimensions and optical properties to simulate light propagation in the normal arterial or breast tissue. Optical properties can be measured with an integrating sphere. The spatial distribution of morphological features associated with vulnerable plaque or breast cancer are estimated using QIA software. This information, along with the IFS basis spectra, are used as input into fluorescence Monte Carlo models to evaluate the ability of different excitation wavelengths to probe morphological structures such as foam cells and necrotic core.

DRS provides information about the presence of Hb, indicative of thrombus or intraplaque hemorrhage, and the amplitude of the scattering coefficient A is related to the presence of foam cells and their depth within the artery wall (superficiality). IFS provides information about fibrous cap thickness through the ratio of MCR components at 340 to 308 nm excitation. Raman spectroscopy also provides information related to the presence of foam cells or necrotic core. Thus MMS modalities provide important diagnostic parameters related to collagen (Raman and IFS)., diffusive scattering (DRS) and NADH (IFS) that are of use for breast cancer diagnosis.

There are additional correlations between IFS and DRS-measured parameters and key morphological features of breast cancer and vulnerable plaque. For example, detection of β-carotene by DRS can be a strong marker of tissue necrosis and extracellular lipid pools. Tryptophan is another fluorophore that plays an important diagnostic role in both atherosclerosis and breast cancer.

Fit coefficients from MMS morphological models can be used to predict disease/tissue parameters using logistic regression. These fit coefficients can be used as parameters for an algorithm for distinguishing vulnerable and non-vulnerable plaque and the full spectrum of breast lesions, both benign and malignant.

Spectroscopic instrumentation for MMS can comprise a combined instrument in which a clinical Raman system and a FastEEM are linked together for use with a single combined spectral probe. Alternatively a smaller integrated clinical instrument for a variety of clinical studies involving atherosclerosis, breast cancer Barrett's esophagus and oral cancer. A number of specialized MMS spectral probes can be used for front-view, sing-viewing and circumferential imaging modes. See for example U.S. application Ser. No. 10/407,923 filed on Apr. 4, 2003, the entire contents of which is incorporated herein by reference. The measurement for breast cancer and atherosclerosis can be obtained using two independent instruments and separate spectral probes. Due to the differences in these probes, which determines the light collection efficiency, it is preferable to use a single probe. This will eliminate registration uncertainties between Raman and DRS/IFS data and ensure that illumination areas will be the same. This instrument provides the full, range of fluorescence excitation wavelengths and can include a front-looking MMS spectral probe.

A combined instrument can use a FastEEM (See U.S. Pat. No. 6,912,412 incorporated herein by reference) and Raman system combined under a single LabView software program that synchronizes the operation of both units. This instrument collects a set of IFS spectra and a DRS spectrum in 0.2 seconds, followed by collection of a Raman spectrum in 1 second, for example. Excitation light from FastEEM and Raman sources is coupled into a single tapered fiber with 0.22 NA. The tapered fiber has a 600 μm core diameter at one end allowing up to four excitation inputs and can be drawn down to a single 200 μm core for use at the distal end of the probe. For ease of fabrication, MMS probes can be assembled with 15 collection fibers surrounding the central excitation fiber. Alternatively a reduced diameter device has 9 fibers around a single fiber in the probe. The 15 fibers are split at the proximal end so that 10 of the 15 fibers are coupled into the Raman spectrograph while the remaining 5 fibers are coupled to the FastEEM spectrograph. The collection fibers have a core diameter of 200 μm with 0.26 NA. High NA Anhydroguide G fibers can be used in the Raman instrument. They are well suited for near IR wavelengths but have a 40-50% transmission loss in the 300-400 nm region. The Superguide G fibers used in FastEEM have negligible transmission losses in the same UV wavelength range, but low NA. In spite of transmission losses in Anhydroguide G fibers, the spectral quality is not significantly reduced, due to the strength of the fluorescence and reflectance signals at these wavelengths. In one embodiment of an MMS probe, both Superguide and Anhydroguide fibers are used in a single probe to provide a baseline performance level with the optimum transmission properties.

Of the three spectral signals (Raman, DRS and fluorescence), Raman is typically the weakest. Thus, a spectral probe capable of collecting high-quality Raman spectra should easily collect fluorescence and reflectance spectra as well. The spectral probe design for the combined instrument is single-ring front-viewing Raman probe.

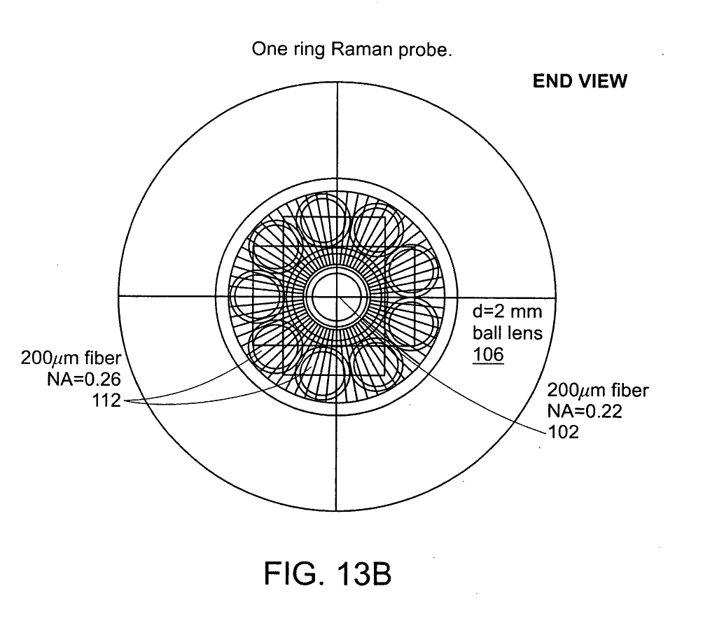

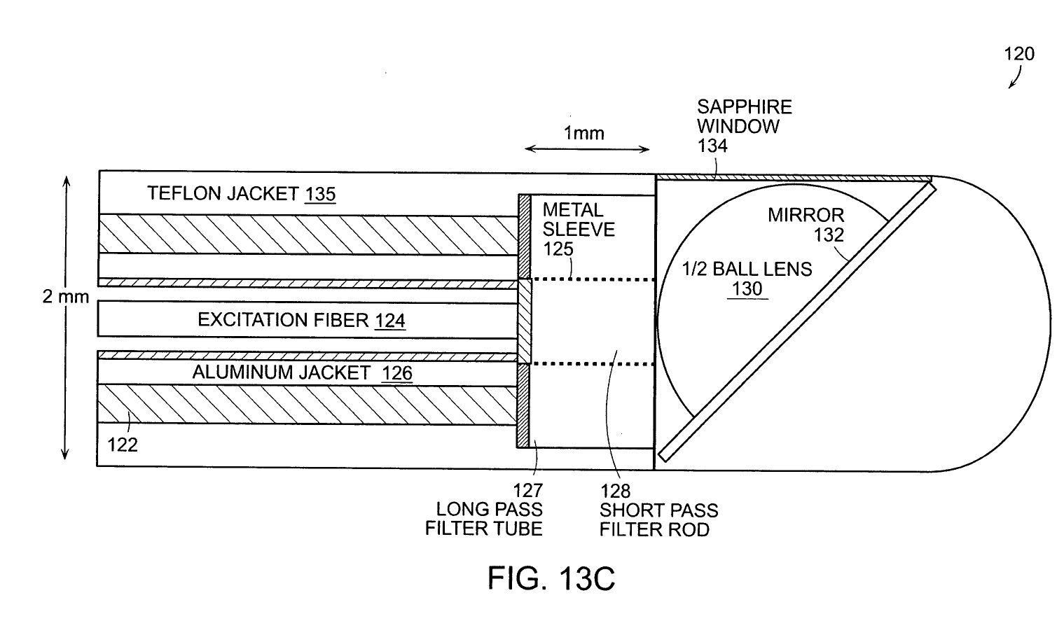

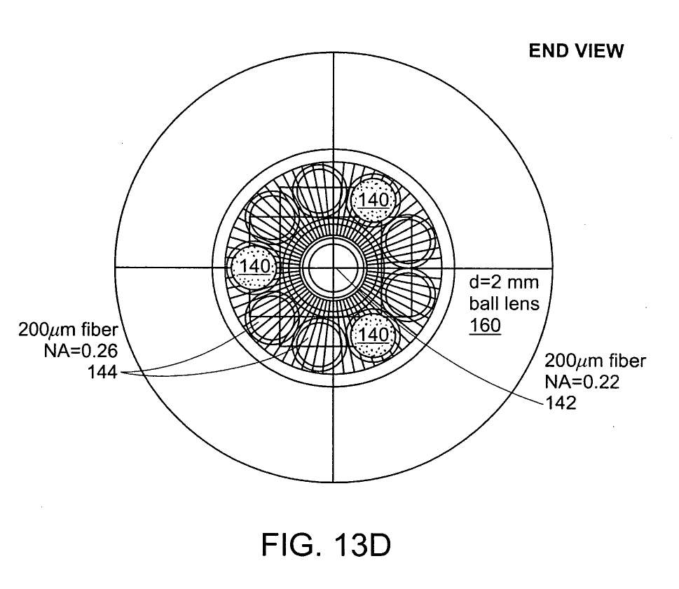

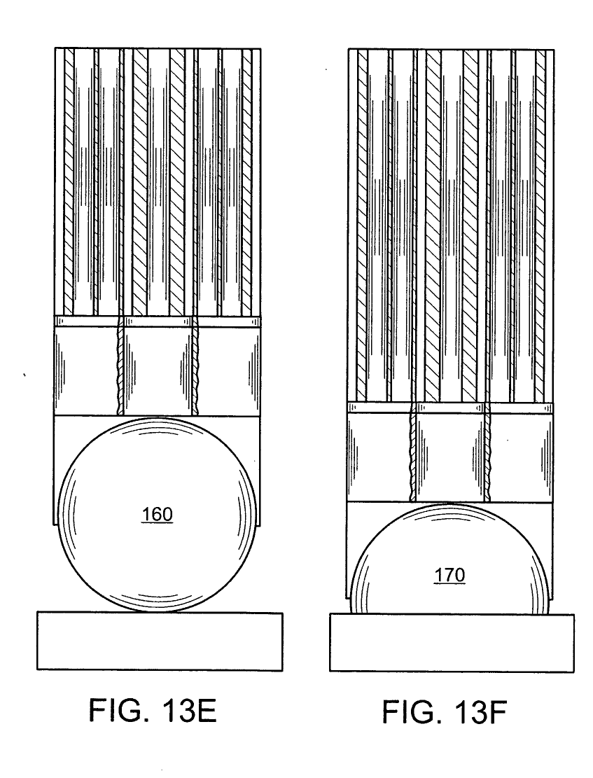

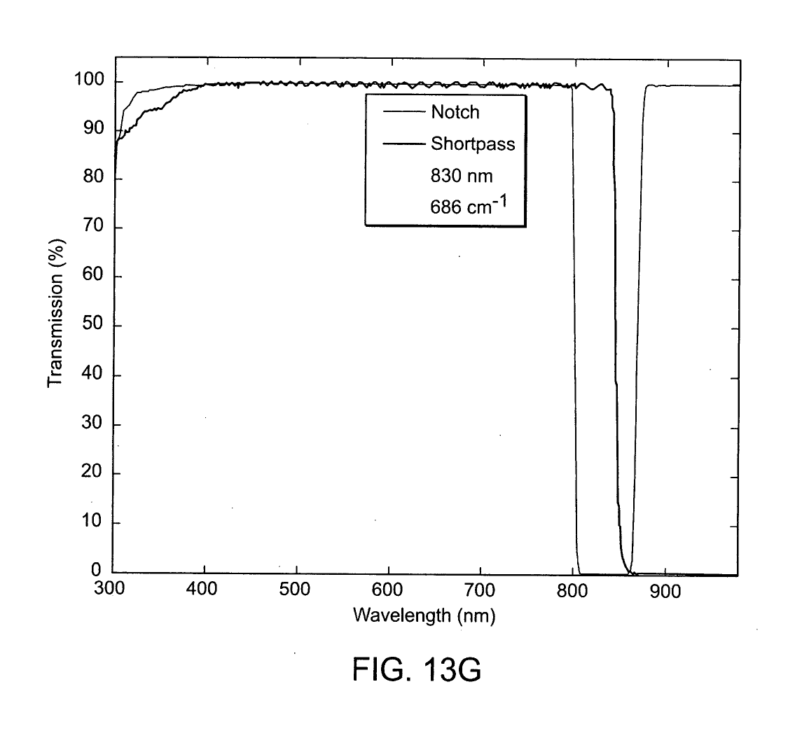

Placement of filters and ball lens, can be the same as the Raman probe, but the filter characteristics has tighter specifications when used with all three spectral modalities. FIG. 13A illustrates the details for a reduced diameter 9-around -1 probe 100 and excitation/collection trajectories through a ball lens 106 that contacts tissue 108. Similar to the Raman probes, the filter module has a filter rod 104 placed on the delivery fiber with transmittance from 300-830 nm and no transmittance (<1%) beyond 850 nm. A filter tube placed on the collection fibers has transmittance from 300-810 nm and from 850-1000 nm and with a narrow 40 nm band centered at 830 nm having low transmittance. An end view of the probe is shown in FIG. 13B with collection fibers 112 positioned in a circular array around central excitation fiber 102. A side looking probe 120 is shown in FIG. 13C in which a half ball lens 130 is in contact with a mirror 132 to reflect light from excitation fiber 124 and filter rod 128 through sapphire window 134. Light returning from the tissue such as artery wall is reflected into collection fibers 122 through long pass filter tube 127. A metal sleeve 125 surrounds filter 128. An aluminum jacket surrounds the excitation fiber 126. A Teflon jacket 135 provides the cylindrical tube that forms the outer wall of the catheter.

In FIG. 13D an end view of a design in which a first group of 3 collection fibers 140 are used to collect reflected light and 3 pairs of fibers 144 collect the Raman light passing through ball lens 160. The central fiber 142 directs light through the forward looking probe with lens 160 in FIG. 13E or half ball lens 170 of FIG. 13F. The filter system used in the probe is shown in FIG. 13G.

The wavelength-dependent sampling volume and depth of penetration of the probe can be determined with tissue phantoms and/or thin sections of tissue. The diameter of the excitation spot illuminating the tissue can be approximately equal for all wavelengths; however, the tissue penetration depth is different for different excitation wavelengths. Because the spot diameter and penetration depth are important for diagnostic algorithms and they are measured and checked with Zemax optical design models and Monte Carlo models.

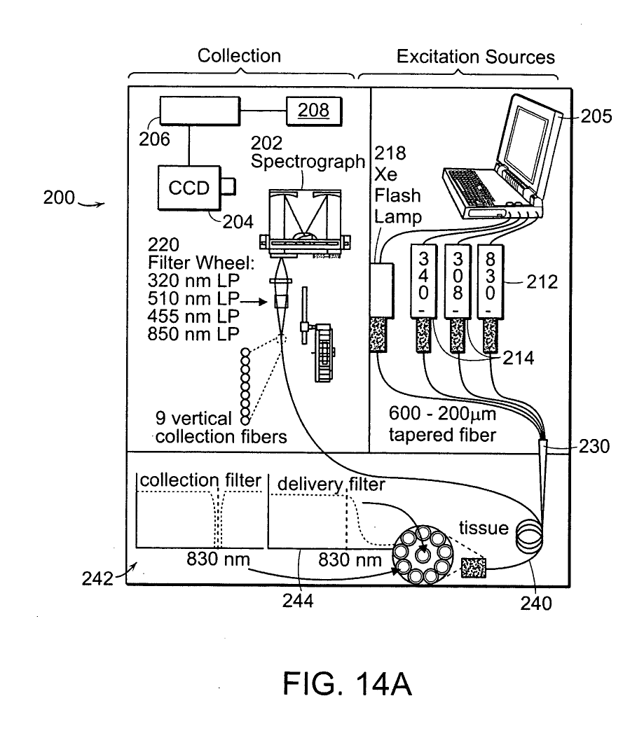

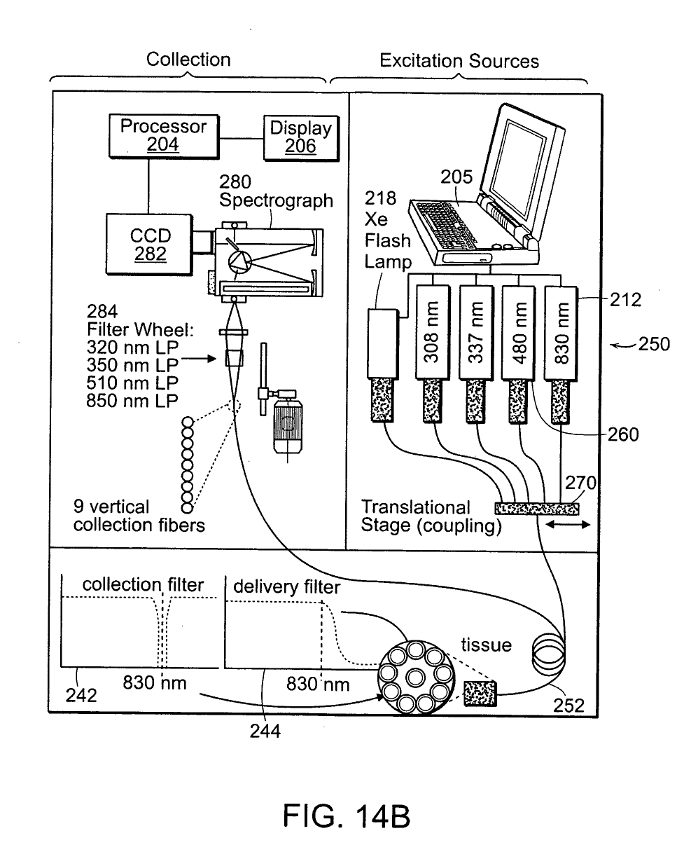

A compact portable MMS instrument that incorporates all three spectroscopic modalities (DRS, IFS and Raman) is shown in FIG. 14A. The fourth modality, LSS, requires no extra instrumentation. A preferred MMS instrument 200 uses solid state light emitting diodes, reducing the instrument size, complexity and cost, and eliminate many maintenance issues related to excimer laser and dye cell operation. The MMS instrument can employ a common spectrograph 202 and CCD 204 for all spectral acquisition.

To accommodate the requirements for using all three spectroscopic modalities, spectra are collected over the wavelength range 300-1000 nm. Excitation light for each modality is delivered sequentially to the sample, and fluorescence, DRS and Raman spectra are acquired. This is followed by real-time analysis of the data, during which IFS spectra are derived from the fluorescence and DRS spectra. The information from the different modalities provides depth-sensitive, complementary chemical and morphological information on tissue sites.

The measurements include IFS spectra excited at 308 and 340 nm, DRS and Raman spectra. The combined TMS/Raman instrument is used for FastEEM fluorescence excitation wavelengths to determine the diagnostic value of the various excitation wavelengths. The most appropriate two or three fluorescence wavelengths can be used in the integrated system.

Data acquisition, analysis and tissue characterization preferably occurs in 5 sec or less. Triggering of the light sources is accomplished by means of a National Instruments Timer/Counter card and a Princeton Instruments CCD controller, respectively. The sequence of operation can be controlled by computer 205 as follows: (1) Initialize CCD for spectral acquisition; (2) open shutter for the CCD and activate insertion of appropriate collection filter; (3) trigger light source (LED, diode laser or flashlamp); (4) acquire spectrum; (5) close shutter; (6) read/transfer data and store in computer 206 and display at 208. The time for acquiring all spectra depends upon the excitation power, thus the exposure time can be adjusted to accommodate signal levels.

Separate excitation and reflectance sources can be used for each spectroscopic modality. Laser emitting diodes 214 (˜1 mW) provide fluorescence excitation light at 308 and 340 nm, a 60W xenon flashlamp generates a continuous spectrum from 300-1000 nm for DRS, and a laser diode 212 at 830 nm (500 mW) will generate the Raman excitation light. A flashlamp 218 can be used in the FastEEM, and the 830 nm laser diode in the Raman system. Each of these four light sources can be focused onto separate 200 μm core diameter optical fibers, and then coupled together into a 600-to-200 μm tapered optical fiber The output can be connected to the combined spectral probe via an SMA connector. The system enables fluorescence excitation wavelengths to be added and/or changed.

UV diode sources can be used compact light sources in the 300-340 nm range available. UV light emitting diodes at wavelengths as short as 275 nm or UV LEDs in the 305-360 nm wavelength range can be used. Present 308 nm LEDs produce 1-2 mW of CW power in a bandwidth of 10-15 nm, emitted from a 0.1 mm aperture over a 30° angular range. Because of this large bandwidth, a filter can be used to restrict the light to a 2 nm bandwidth. Thus, under present conditions, ˜1 μJ of 308 nm light can be delivered via 200 micron core, 0.26 NA, fused silica optical fiber in 10 ms, resulting in the acquisition of high SNR fluorescence spectra. Characteristics of 340 nm LEDs are even more favorable.

Each of the spectral probe collection fibers, typically nine, (fifteen in one design) are coupled to an SMA connector mounted on the front panel of the instrument. Long (wavelength) pass filters 220 mounted on a programmable wheel driven by a stepper motor are positioned in the return beam path to prevent Raman and fluorescence excitation light scattered from the tissue from entering the spectrograph. Since the reflectance measurements cover a broad range (300-1000nm), the acquired spectra contain second order contributions. Taking two reflectance measurements, one with no filter and another with a long pass 500 nm cutoff filter (mounted on the wheel), eliminates these contributions. The unfiltered reflectance provides spectral information below 600 nm, and the filtered reflectance provides information above 500 nm. The Princeton Instruments Spec10:400BR CCD camera of the Raman system can be coupled to an Acton Research Spectra Pro 150 spectrograph with a grating blazed at 500 nm and 200 grooves/mm. Alternatively two separate gratings or dispersive elements can deliver different light modalities onto separate regions of the detector.

This combination of fluorescence, reflectance and Raman capabilities in one instrument provides a compact clinical instrument. With a single spectrograph/CCD combination, a spectral range of 300-1000 nm is covered, compared to 155 nm in our existing Raman system. This causes an increase in spectral dispersion by a factor of 4.5, and a reduction in system resolution from 10 to 45 cm⁻¹. However, if the spectral resolution degrades the accuracy of the Raman fit coefficients significantly such that diagnostic accuracy is also degraded. A two spectrograph/CCD system can also be used with one spectrograph/CCD combination is dedicated to Raman while the other to fluorescence/reflectance. A high-speed mirror will direct the collected light to appropriate spectrograph/CCD combination.

A further embodiment of a system 250 is shown in FIG. 14B in which a translational stage 270 is used to couple light from the source sequentially into the probe 252. This contrasts with the prior embodiment where the sources are coupled to probe 240 with combiner 230 to provide simultaneous illumination. The delivery 244 and collection 242 filters are shown schematically. Another source 260 is also used and accounted for in the filter which 284, spectrograph 280 and detector 282 system.

The detection of vulnerable plaques, margin assessment in breast cancer and transdermal needle biopsies can be performed using front-viewing, side-viewing or circumferential imaging probes.

Using the integrated MMS system, spectra are collected from several of these margins prior to excision and thus only tissue that would normally be excised during the procedure will be removed. During each procedure, the distal end of the sterilized MMS front-viewing probe is placed in gentle contact with the marginal breast tissue in the surgical cavity under direct visualization while spectra are acquired. All room and surgical lights will be turned off during the measurements. The spectrally examined marginal tissue will then excised by the surgeon and submitted for conventional pathological examination.

Under local anesthesia following a manual incision of the skin, a cannula having a diameter 0.5 to 2 cm is advanced toward the suspect lesion guided under ultrasound or stereotactic mammography. The central channel of the needle contains a circular blade that is used to cut the biopsy and will provide access for the MMS probe. Once positioned in the lesion, a MMS side-viewing probe is inserted in the central channel and acquire a series of spectra as the probe is withdrawn along the opening. The probe is withdrawn and cutting blade replaced and a biopsy is acquired. Biopsies are performed over a 360 degree around the axis of the needle without it being withdrawn with typically twelve cores of tissue are removed using 11 to 14 gauge needles. The excised biopsy specimens are submitted for specimen. radiography to document the presence of calcification and then conventional pathology.

A digital photograph of the lesion and probe placement is recorded. Precise registration between the probe location and biopsy site is ensured by immediately scoring a circular region of tissue slightly larger than diameter of the probe with a 1.5 mm punch biopsy. A larger punch biopsy (˜-3.5 mm) is used to remove a larger tissue specimen for histopathology and slide preparation. The smaller mark is located later when viewing the slide under the microscope.

While the present invention has been described here in conjunction with a preferred embodiment, a person with ordinary skill in the art, after reading the foregoing specification, can effect changes, substitutions of equivalents and other types of alterations to the system and method that are set forth herein. Each embodiment described above can also have included or incorporated therewith such variations as disclosed in regard to any or all of the other embodiments. Thus, it is intended that protection granted by Letters Patent hereon be limited in breadth only by definitions contained in the appended claims and any equivalents thereof.

What is claimed is:

1. A system for spectroscopic measurement of tissue comprising:
a light source providing light for Raman and fluorescence collection;
a probe that delivers light onto tissue; and
a detector that detects Raman and fluorescent light from the tissue.

2. The system of claim 1 further comprising a data processing system.

3. The system of claim 2 wherein the processing system processes reflectance data detected by the detector.

4. The system of claim 1 wherein the probe comprises a plurality of optical fibers and distally mounted filters.

5. The system of claim 1 wherein the light source comprises a Raman excitation light source and a fluorescence excitation light source.

6. The system of claim 1 wherein the detector detects a reflectance spectrum.

7. The system of claim 6 wherein the light source further comprises a broadband light source for obtaining the reflectance spectrum.

8. The system of claim 1 wherein the probe comprises at least one excitation optical fiber coupled to the light source and a plurality of collection optical fibers.

9. The system of claim 8 wherein the collection optical fibers are optically coupled to a spectrograph which disperses the collected light for detection by the detector.

10. The system of claim 1 wherein the probe comprises a flexible catheter having a side-looking distal end.

11. The system of claim 1 wherein the probe has a ball lens on a distal end.

12. The system of claim 8 wherein the excitation optical fiber has a first filter and the collection optical fibers have a second filter.

13. The system of claim 1 wherein the detector detects Raman fluorescence and reflected light.

14. The system of claim 1 wherein the probe comprises an endoscope.

15. The system of claim 1 wherein the probe has a diameter for insertion through an endoscope channel.

16. The system of claim 2 wherein the processing system determines a size of a cellular structure in tissue.

17. The system of claim 1 further comprising coupling the collected Raman light to a first dispersive element and coupling the collected fluorescence light to a second dispersive element.

18. The system of claim 17 wherein the first dispersive element couples light to a first detector region and the second dispersive element couples light to a second detector region.

19. The system of claim 4 wherein the distally mounted filters include a short pass filter at a distal end of a light delivery fiber and a long pass filter at a distal end of a collection fiber.

20. The system of claim 1 wherein the light source includes a Raman excitation light source emitting light in a range between 750 nm and 1000 nm and further includes a fluorescence source emitting between 300 nm and 500 nm.

21. A system for-spectroscopic measurement of tissue comprising:
a light source providing light for Raman and reflectance collection;
a probe that delivers light onto tissue; and
a detector that detects Raman and reflected light from the tissue.

22. The system of claim 21 further comprising a data processing system.

23. The system of claim 22 wherein the processing system processes fluorescence data detected by the detector.

24. The system of claim 21 wherein the probe comprises a plurality of optical fibers and distally mounted filters.

25. The system of claim 21 wherein the light source comprises a Raman excitation light source and a broadband excitation light source.

26. The system of claim 21 wherein the detector detects a reflectance spectrum.

27. The system of claim 23 wherein the light source further comprises plurality of laser diodes for obtaining a fluorescence spectrum.

28. The system of claim 21 wherein the probe comprises at least one excitation optical fiber coupled to the light source and a plurality of collection optical fibers.

29. The system of claim 28 wherein the collection optical fibers are optically coupled to a spectrograph which disperses the collected light for-detection by the detector.

30. The system of claim 21 wherein the probe comprises a flexible catheter having a side-looking distal end.

31. The system of claim 21 wherein the probe has a ball lens on a distal end.

32. The system of claim 28 wherein the excitation optical fiber has a first filter and the collection optical fibers have a second filter.

33. The system of claim 1 wherein the probe comprises an endoscope.

34. The system of claim 21 wherein the probe has a diameter for insertion through an endoscope channel.

35. The system of claim 22 wherein the processing system determines a size of a cellular structure in tissue.

36. The system of claim 21 further comprising coupling the collected Raman light to a first dispersive element and coupling the collected reflected light to a second dispersive element.

37. The system of claim 36 wherein the first dispersive element couples light to a first detector region and the second dispersive element couples light to a second detector region.

38. The system of claim 21 wherein the distally mounted filters include a short pass filter at a distal end of a light delivery fiber and a long pass filter at a distal end of a collection fiber.

39. The system of claim 23 wherein the light source includes a Raman excitation light source emitting light in a range between 750 nm and 1000 nm and further includes a fluorescence source emitting between 300 nm and 500 nm.

40. The system of claim 22 further comprising a processing system for measuring arterial plague.

41. The system of claim 22 wherein the system measures cellular structure for cancer diagnosis.

42. A method for spectroscopic measurement of a material comprising:
providing a light source system for Raman and fluorescence excitation light;
illuminating a material with light from the light source system; and detecting Raman and fluorescent light from the material.

43. The method of claim 42 further comprising processing spectral data detected by the detector with a processing system.

44. The method of claim 42 further comprising processing reflectance data detected by the detector.

45. The method of claim 42 further comprising providing a probe having a plurality of optical fibers and distally mounted filters.

46. The method of claim 42 further comprising providing a light source having a Raman excitation light source and a fluorescence excitation light source.

47. The method of claim 42 further comprising providing a broadband light source for obtaining a reflectance spectrum.

48. The method of claim 42 further comprising providing a probe having at least one excitation optical fiber coupled to the light source and a plurality of collection optical fibers.

49. The method of claim 48 further comprising coupling the collection optical fibers to a spectrograph which disperses the collected light for detection by the detector.

50. The method of claim 42 further comprising providing a flexible catheter having a side-looking or forward looking distal end.

51. The method of claim 42 further comprising detecting Raman fluorescence and reflected light.

52. The method of claim 50 further comprising inserting the probe through an endoscope channel.

53. The method of claim 42 further comprising determining a size of a cellular structure in tissue.

54. The method of claim 42 illuminating tissue with a Raman excitation light source emitting light in a range between 750 nm and 1000 nm and illuminating the tissue with a fluorescence source emitting between 300 nm and 500 nm.

55. The method of claim 42 wherein the method comprises measuring a tissue sample removed from a body.

56. A method for spectroscopic measurement of a material comprising:
providing a light source for Raman and reflectance light delivery;
illuminating the material with light; and
detecting Raman and reflected light from the material.

57. The method of claim 56 further comprising processing Raman and reflectance spectra of tissue with a data processor.

58. The method of claim 56 further comprising providing a Raman excitation light source and a fluorescence excitation light source.

59. The method of claim 57 further comprising providing a broadband light source for obtaining the reflectance spectrum.

60. The method of claim 56 further comprising providing a probe having at least one excitation optical fiber coupled to a light source and a plurality of collection optical fibers.

61. The method of claim 56 further comprising illuminating tissue with light from a plurality of light sources in sequence with a single light delivery probe.

62. The method of claim 56 further comprising simultaneously collecting Raman and reflected light from tissue.
